(19) United States
(12) Patent Application Publication
(10) Pub. No.: US 2019/0046348 A1
(43) Pub. Date: Feb. 14, 2019

(54) Back-up colostomy bag

(71) Applicant: Leroy Angelle, Lafayette, LA (US)

(72) Inventor: Leroy Angelle, Lafayette, LA (US)

(21) Appl. No.: 16/101,166

(22) Filed: Aug. 10, 2018

Related U.S. Application Data
(60) Provisional application No. 62/543,906, filed on Aug. 10, 2017.

(51) Int. Cl. A61F 5/449; A61F 5/44

(52) U.S. Cl. CPC A61F 5/449; A61F 5/4404

(57) ABSTRACT

The invention is a back-up colostomy bag. The bag is waterproof and flexible, and provides a drawstring mouth which secures around the flange to contain any leakage. In the event that the standard colostomy bag becomes detached from the flange, any leakage is fully contained and may be easily rinsed out later.

### CROSS-REFERENCE TO RELATED APPLICATIONS

This application relates back to, and claims the priority of Provisional Patent Application No. 62/543,906 filed on Aug. 10, 2017.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to medical equipment and supplies, and in particular to a back-up colostomy bag. For most survivors of colon cancer, wearing a colostomy bag for the collection and disposal of feces is an unpleasant fact of life. The bag must be compatible with the user's skin surrounding the ostomy, so a separate flange is applied. Unfortunately, the seal is not perfect and occasionally the flange becomes separated from the colostomy bag. The colostomy bag is concealed under the user's clothing, and held in position by an elastic strap or similar device, so the user usually does not know that it has become disconnected. The result, too frequently, is a terrible, smelly mess that creates a substantial disease risk and must be cleaned up immediately. If a device could contain such leaks quickly, easily, and securely until the user notices that the colostomy bag has become disconnected, such a device would be very useful, and it would be well received.

A search of the prior art reveals various devices which have been developed to provide the features of a colostomy bag with additional features. In particular, none of the prior art devices provide containment of a leaking colostomy bag. None are closely related to the present invention, but several include features which resemble those of the present invention. Each has proven to be less than satisfactory for the present purpose in its own way.

One-piece ileostomy or colostomy bag connector, U.S. Pat. No. 5,098,420 (priority Nov. 12, 1987), provides a one-piece bag connector for an ileostomy bag, colostomy bag or the like. The connector comprises an annular body portion, lip portion and a belt attachment. The body portion is adapted to surround a stoma of a user and the lip portion is connectable to a bag such that material may be discharged from the stoma to the bag without leakage. The body portion is made of resilient material and directly contacts the user thus avoiding the use of any gels or adhesive materials. The belt attachment connects a belt to the body portion such that a slight pressure is exerted against the user. This pressure plus the resilient nature of the body portion assures a proper seal between the user and the bag connector. Further, an easily attachable/detachable snap or screw connection is provided between the lip portion and the bag to assure a proper seal as well as convenience and ease in changing and/or removing the bag. The annular body portion, lip portion and belt attachment can be molded from plastic or the like to form the one-piece connector.

Colostomy bag belt, U.S. Pat. No. 5,947,942 (priority Jul. 18, 1997), provides a colostomy bag belt which utilizes: an inner comfort belt which holds a skin patch against the wearer's skin, the skin patch being connected to a connector forming a conduit through the comfort belt; an exterior belt which forms a pouch therein and has a leak-proof safety pouch within the belt and has a connector forming a conduit through the exterior belt and into the safety pouch and which has a sealable opening through which as colostomy bag can be inserted into the safety pouch and secured to the connector; a flexible intermediate connector which completes the conduit from the skin patch to the safety pouch; and a colostomy bag placed in the safety pouch and connected to the conduit to receive the waste which exits the stoma and passes through the conduit.

Ostomy bag cover, U.S. Pat. No. 5,607,412 (priority Jun. 6, 1995), provides an ostomy bag cover which is formed of a single piece of a soft knit fabric, folded and sewn so that there are no rough edges in contact with the patient. The cover contains the ostomy bag and isolates the patient from direct contact of the plastic bag with the skin. The cover has a distal side panel and left and right proximal side panels that meet at a vertical midline on the patient side of the cover. The panels are left detached from one another from the top seam partway down the midline to define a vertical slot through which the fitting or flange of the ostomy bag protrudes. Another slot is provided along the bottom seam to accommodate a drain tube.

Ostomy bag with multi-stage filter, U.S. Pat. No. 5,306,264 (priority Jan. 14, 1993), provides an ostomy bag which includes a multi-stage filter system that provides contamination protection for a deodorizing filter in the system. The multi-stage filter system also includes a gas transmissible protection filter that is impassible to semi-liquid waste material. The protection filter is located in the ostomy bag to precede the deodorizing filter such that gaseous waste must pass through the protection filter before it passes through the deodorizing filter. In one embodiment of the invention wherein the ostomy bag has a single chamber, the protection filter is a two layer structure with different pore counts that overlay the deodorizing filter. In another embodiment of the invention wherein the ostomy bag has dual chambers, the protection filter, without overlaying the deodorizing filter, is disposed across the flow path of gaseous waste to the deodorizing filter such that any gaseous waste that reaches the deodorizing filter must flow through the protection filter. Thus semi-liquid waste material cannot contact the deodorizing filter since it cannot bypass the protection filter to reach the deodorizing filter. (See also U.S. Pat. No. 5,643,234).

Ostomy bag, U.S. Pat. No. 5,549,587 (priority Jun. 7, 1995), provides an ostomy bag having a liquid-gas separation device includes a separator disposed within a flexible film pouch used as an ostomy bag. The separator is made of an absorbent material to collect liquids and separate these components from any gas entering the ostomy bag through the stomal opening thereof. The separator is spaced from a filter incorporated in the ostomy bag to further enhance gas-liquid separation. The separator may be disposed in the bag in an attached or unattached configuration. A perforated wall may be included within the flexible film pouch to minimize clogging of the separator by solids so as to improve and gas-liquid separation. An absorbent pad covers the filter to absorb any fluid surging over the separation and flowing through the filter.

Continent ostomy port, U.S. Pat. No. 6,465,476 (priority Feb. 25, 1998), provides a continent ostomy port device, having a face plate defining a selectively sealable aperture which is alignable with the opening of a stoma formed in the body of a user of the device. A closure portion is adapted to permit selective and repeatable covering and uncovering of the aperture in the face plate. A catheter portion extends from one side of the face plate and extends proximally, and one end of the catheter portion is disposed interior of the user's body, within the ostomy site, when the port device is in normal use position. The catheter portion has continuous exterior and interior side walls, the latter defining a major lumen. The catheter portion is sized and shaped appropriately for non-surgical installation through a stoma to a sufficient distance that the presence of the catheter portion within the stoma provides a physical barrier which reduces the incidence of stoma prolapse, without the use of extraneous, externally applied materials or additional surgery. Retaining structure is connected to the catheter of the continent ostomy port and is non-surgically, snugly fittable into the stoma, to cause the port device to be self-retaining in a normal use position within a stoma of the user, without the need for special surgery or extraneous, external fixation materials such as tape, belts, and adhesives. The retaining structure is also usefully connected to other medical catheters.

An effective system for containing leaks from a colostomy bag would be very useful and would be well received. A back-up colostomy bag, providing a drawstring mouth which secures around the flange to contain any leakage, would resolve this problem.

### SUMMARY OF THE INVENTION

Accordingly, the invention is a back-up colostomy bag. The bag is waterproof and flexible, and provides a drawstring mouth which secures around the flange to contain any leakage. In the event that the standard colostomy bag becomes detached from the flange, any leakage is fully contained and may be easily rinsed out later.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate two embodiments of the invention and, together with the description, serve to explain the principles of the invention.

FIG. 1 is a side view of the first exemplary embodiment, displaying the back-up colostomy bag 10, and the drawstring mouth 11.

FIG. 2 is a front perspective view of the first exemplary embodiment with a standard colostomy bag inserted, displaying the back-up colostomy bag 10, the drawstring mouth 11, the standard colostomy bag 12, and the flange 14.

FIG. 3 is a side perspective view of the second exemplary embodiment, displaying the back-up colostomy bag 10, and the affixed flange 10A.

FIG. 4 is a front perspective view of the first exemplary embodiment, displaying the back-up colostomy bag 10, and the drawstring mouth 11.

FIG. 5 is a top view of the first exemplary embodiment with a standard colostomy bag, displaying the back-up colostomy bag 10, the drawstring mouth 11, the standard colostomy bag 12, the cap 13, and the flange 14.

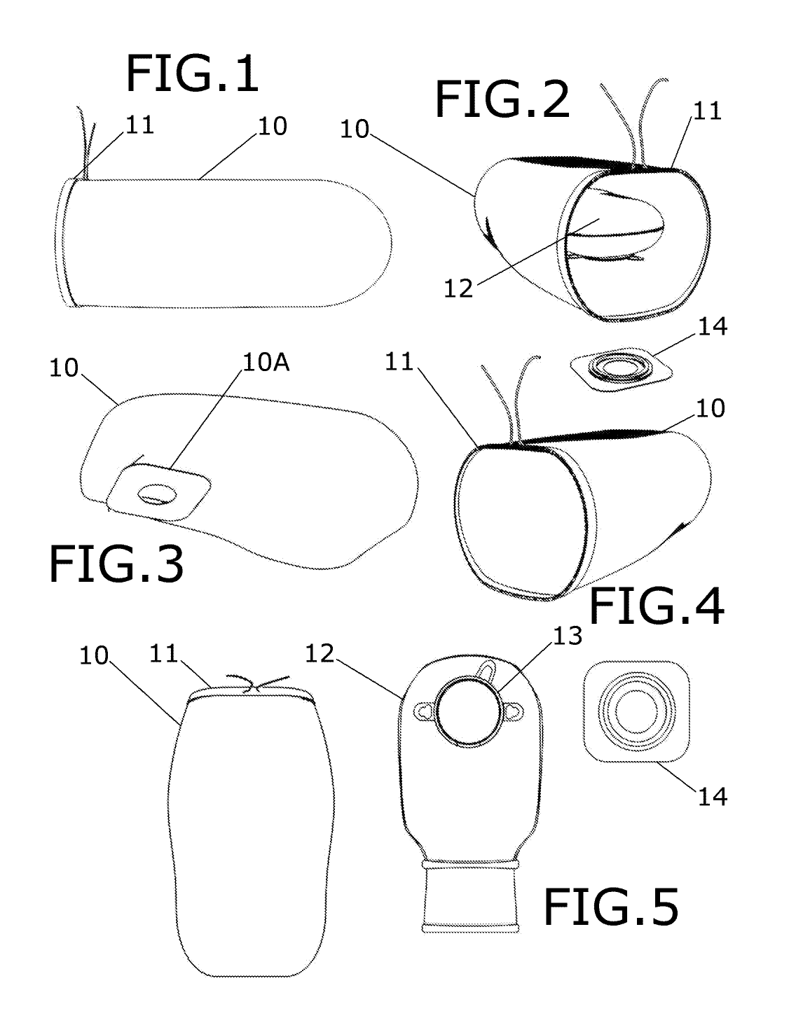

### DETAILED DESCRIPTION OF THE INVENTION

Referring now to the invention in more detail, the invention is a back-up colostomy bag 10.

It is to be understood that while the invention is intended for use with a standard colostomy bag 12, this is not intended as a limitation. With suitable modification, the invention may be used with other objects which are secured to each other around the edge of a flange 14 or similar device, and are at risk of becoming separated.

The first exemplary embodiment is comprised of a back-up colostomy bag 10 which is waterproof and flexible, and provides a drawstring mouth 11 which secures around the flange 14 to contain any leakage. In the event that the standard colostomy bag 10 becomes detached from the flange 14, any leakage is contained and may be easily rinsed out later.

To use the first exemplary embodiment, the user may insert the standard colostomy bag 12 into the back-up colostomy bag 10, and affix the drawstring mouth 11 around the flange 14. The user may then secure the combined bags to the user's body with an elastic strap or similar device.

The second exemplary embodiment is similar in structure and function to the first exemplary embodiment, with the following modifications. The back-up colostomy bag 10 is designed to be used with a different model of standard colostomy bag 12. A permanently affixed flange 10A is provided instead of a drawstring mouth 11.

To use the second exemplary embodiment, the user may insert the standard colostomy bag 12 into the back-up colostomy bag 10, and affix the affixed flange 10A against the flange 14 before securing the standard colostomy bag 12 to the flange 14. The user may then secure the combined bags to the user's body with an elastic strap or similar device.

The back-up colostomy bag 10 and the drawstring mouth 11 are preferably manufactured from a flexible, durable material which provides substantial structural strength and is easily cleaned, such as plastic. The affixed flange 10A is preferably manufactured from a rigid, durable material such as cardboard. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A back-up colostomy bag which is waterproof and flexible, and provides a drawstring mouth which secures around the flange to contain any leakage from a standard colostomy bag, such that in the event that the standard colostomy bag becomes detached from the flange, any leakage is contained and may be easily rinsed out later.

2. The back-up colostomy bag of claim 1, wherein the user may insert the standard colostomy bag into the back-up colostomy bag, and affix the drawstring mouth around the flange, then secure the combined bags to the user's body with an elastic strap or similar device.

3. The back-up colostomy bag of claim 1, wherein the back-up colostomy bag is designed to be used with a different model of standard colostomy bag, providing a permanently affixed flange instead of a drawstring mouth.

4. The back-up colostomy bag of claim 2, wherein the user may insert the standard colostomy bag into the back-up colostomy bag, and affix the affixed flange against the flange before securing the standard colostomy bag to the flange, then secure the combined bags to the user's body with an elastic strap or similar device.

5. The back-up colostomy bag of claim 2, wherein the back-up colostomy bag and the drawstring mouth are preferably manufactured from a flexible, durable material which provides substantial structural strength and is easily cleaned, such as plastic.

6. The back-up colostomy bag of claim 1, wherein the affixed flange is preferably manufactured from a rigid, durable material such as cardboard.
